(19) United States
(12) Patent Application Publication
(10) Pub. No.: US 2004/0142405 A1
(43) Pub. Date: Jul. 22, 2004

(54) Method of detecting drug resistance

(75) Inventors: Lital Alfonta, Rehovot (IL); Immanuel Blumenzweig, Bat Yam (IL); Maya Zayats, Nazereth Ilite (IL); Lea Baraz, Jerusalem (IL); Moshe Kotler, Mevasseret Zion (IL); Itamar Willner, Mevasseret Zion (IL)

Correspondence Address: BROWDY AND NEIMARK, P.L.L.C., 624 NINTH STREET, NW, SUITE 300, WASHINGTON, DC, 20001-5303, US

(73) Assignee: YISSUM RESEARCH DEVELOPMENT COMPANY OF THE HEBREW UNIVERSITY OF JERUSALEM, Jerusalem (IL)

(21) Appl. No.: 10/348,326

(22) Filed: Jan. 22, 2003

(51) Int. Cl. C07K 14/00; C12Q 1/00; C12Q 1/37

(52) U.S. Cl. 435/23; 435/4; 530/350

(57) ABSTRACT

A method for detecting a drug-resistant virus, whose resistance is due to mutations in a viral protease which normally cleaves a substrate into at least two segments. The method comprises providing an electronic transducer having bound thereto a first binding molecule capable of specifically binding a first segment of the substrate and providing a second binding molecule capable of specifically binding a second segment of the substrate, the second binding molecule being capable of bringing about the precipitation of a precipitate on the electronic transducer. Detection of the presence or absence of the precipitate on the electronic transducer indicates the presence or absence of inhibitory activity of the drug. A screening method for potential viral drugs is also disclosed.

### FIELD OF THE INVENTION

This invention relates to a method of detecting drug resistance, and particularly drug resistant proteases. The invention also relates to inhibitors of protease activity and methods of identifying substances capable of such inhibition.

### BACKGROUND OF THE INVENTION

The following publications may be relevant to understanding the background to the invention:

1. Hertogs, K. et al. Phenotypic and genotypic analysis of clinical HIV-1 isolates reveals extensive protease inhibitor cross-resistance: a survey of over 6000 samples. Aids 14, 1203-1210 (2000).

2. Nillroth, U. et al. Human immunodeficiency virus type 1 proteinase resistance to symmetric cyclic urea inhibitor analogs. Antimicrob. Agents Chemother 41, 2383-2388 (1997).

3. Garcia-Lerma, J. G. & Heneine, W Resistance of human immunodeficiency virus type 1 to reverse transcriptase and protease inhibitors: Genotypic and phenotypic testing. J. Clin. Virol. 21, 197-212 (2001).

4. Willner, I. & Willner, B. Biomaterials integrated with electronic elements: en route to bioelectronics. Trends Biotechnol. 19, 222-230 (2001).

5. Alfonta, L., Wiflner, I., Throckmorton, D. J. & Singh, A. K. Electrochemical and quartz crystal microbalance detection of the cholera toxin employing horseradish peroxidase and GM1-functionalized liposomes. Anal. Chem. 73, 5287-5295 (2001).

6. Patolsky, F, Lichtenstein, A., Kotler, M. & Willner, I. Electronic transduction of polymerase or reverse transcriptase induced replication processes on surfaces: Highly sensitive and specific detection of viral genomes. Angew. Chem. Int. Ed. 40, 2261-2265 (2001).

7. U.S. Pat. No. 5,942,388 (Willner et al).

8. WO 97/04314 (Willner et al).

The above references will be acknowledged in the text below by indicating their numbers from the above list.

Proteases belong to an important group of enzymes which serves widely varied functions in the biosphere. The modification of protease activity can have important therapeutic implications. For example, some drugs function by binding to proteases, thereby inhibiting undesirable protease activity. Drug resistance may occur when mutations in the protease structure reduce or eliminate the affinity of the drug to the protease.

Retroviral viruses, such as the human immunodeficiency virus (HIV) which causes AIDS, contain genonmic RNA which encodes Gag, Gag-Pol and Env polyproteins that are subsequently cleaved to mature viral proteins. The Gag and Gag-Pol polyproteins are translated from mRNA that is indistinguishable from the full-length viral genomic RNA. The viral Gag and Gag-Pol polyproteins are processed by a virus encoded protease (PR) which is one of the individual proteins making up the uncleaved polyprotein. Cleavage of the viral polyproteins is a key step in viral maturation; without specific cleavage of the precursors, the virion is not infectious. Previous studies have indicated that PR is active in its Gag-Pol precursor form, and that the cleavage of the polyproteins may occur by inter- or intra-molecular mechanisms.

HIV PR inhibitors are effective against wild type HIV both in vitro and in vivo. However, use of such inhibitors in anti AIDS treatment resulted in a rapid selection of HIV variants selected for displaying reduced susceptibility to the PR inhibitors. Currently, most of the clinically approved inhibitors of HIV PR are peptide mimetics, which interact with the protease active site and adjacent substrate specificity pockets. Mutations, both in these regions and in distal sites of the protease, affect the inhibitor and substrate binding by altering the number and/or strength of subsite interactions. Consequently, in the presence of a PR inhibitor, there is a replicative advantage for HIV drug-resistant mutants, which have a decreased affinity to the inhibitor while retaining sufficient enzyme activity to process the Gag and Gag-Pol polyproteins, thus enabling release of infectious virus.

The currently used methodology to detect drug resistant viruses in AIDS patients consists principally of phenotyping and genotyping viruses isolated from blood samples.¹ Phenotyping of the viral PRs is carried out by the isolation and propagation of viruses in cultured cells in the presence of anti HIV drugs, or by the cloning and expression of the viral PR in bacterial cells, and assessing the enzyme activity in the presence of protease inhibitors (PIs).² Genotypic assays detect the mutations responsible for PR-resistance by sequencing the PR-encoding region in the viral RNA or by point mutations assays.³

Bioelectronics, and specifically, the development of biosensors is a rapidly developing research field.⁴ Electronic biosensors transduce biorecognition events into electronic signals. Electrodes, piezoelectric crystals and field-effect transistors are often used as electronic transduction units. Electrochemical transduction of enzyme-substrate interactions, antigen-antibody or nucleic acid-DNA recognition processes, have been described. Microgravimetric, quartz-crystal-microbalance measurements were employed for the detection of antigens, antibodies or DNA, and field-effect transistors were used to analyze antibody-antigen complexes. Recent efforts in bioelectronics were directed towards the amplified detection of antigen-antibody or nucleic acid-DNA interactions. The replication of the analyzed DNA to yield a redox-active replica that is coupled to a bioelectrocatalytic cascade, or the conjugation of nucleic acid-labeled particulate systems such as liposomes or nanoparticles were used to amplify DNA detection processes.

A powerful method to amplify antigen-antibody or DNA (RNA) detection involves the coupling of a biocatalytic conjugate to the bio-recognition complex that results in the precipitation of an insoluble product on electronic transducers.^{5,6} The insulation of the electrodes by the insoluble product, or the increase of the mass associated with piezoelectric crystals as a result of the formation of the precipitate are electronically transduced.

U.S. Pat. No. 5,942,388⁷ discloses an electrobiochemical system for the determination of the presence of an analyte in a liquid medium. The system comprises an electrode having immobilized thereon one member of a recognition pair such as an antibody-antigen or ligand-receptor pair, the other member being the analyte. The presence of the analyte in the medium results in the formation of a complex immobilized to the electrode. The system also comprises redox molecules capable of changing their redox state by accepting electrons from or donating electrons to the electrode. The formation of the complex causes a change in the electrical response of the system.

WO 97/04314⁸ discloses a system for determining binding between two members of a recognition pair. The system comprises a probe containing a piezoelectric crystal with electrodes on two opposite faces of the crystal. A first member of the recognition pair is immobilized to the electrodes. Binding of the second member of the pair to the first member, or dissociation of the second member from the first member causes a change in mass on the crystal which results in a change of the probe's resonance frequency.

### SUMMARY OF THE INVENTION

It is an object of the present invention to provide a method for detecting drug-resistant viruses.

It is a further object of the invention to provide a method for screening potential protease inhibitor compounds.

In a first aspect of the invention, there is provided a method for detecting whether a tested chemical compound has the ability to inhibit the activity of a protease, the protease being capable of specifically cleaving a substrate into at least two segments, the method comprising:

(a) providing an electronic transducer having bound thereto a first binding molecule capable of specifically binding a first segment of said substrate;

(b) providing a second binding molecule capable of specifically binding a second segment of said substrate, said second binding molecule being capable of bringing about the precipitation of a precipitate on said electronic transducer;

(c) incubating said substrate with said protease in the presence and in the absence of the chemical compound;

(d) incubating the electronic transducer with the substrate of step (iii);

(e) incubating the electronic transducer of step (iv) in the presence of said second binding molecule;

(f) incubating the electronic transducer of step (v) under conditions which bring about the precipitation of the precipitate on said electronic transducer; and

(g) detecting the presence or absence of the precipitate on said electronic transducer, an increase in the amount of precipitate in the presence of said chemical compound indicating inhibition of the activity of the protease by the compound.

In a preferred embodiment of this aspect of the invention, the method further comprises (a) providing a third binding molecule capable of specifically binding the first segment of the substrate at a site other than the site bound by the first binding molecule, the third binding molecule being capable of bringing about the precipitation of a precipitate on the electronic transducer; (b) incubating the electronic transducer of step (iv) in the presence of the third binding molecule and (c) detecting the presence or absence of the precipitate on the electronic transducer, the presence of the precipitate indicating that the first binding molecule has bound the first segment.

In a preferred embodiment, one or more of each of the binding molecules is an antibody. The substrate may be any molecule capable of being cleaved into at least two segments by a protease. In the present specification, the term “segments” refers to portions of the substrate obtained on cleavage of the substrate by a protease. Preferably, the substrate is a polyprotein which is cleaved into its individual protein components.

Examples of an electronic transducer useful in the invention include an electrode or a quartz crystal, preferably an Au-electrode or an Au-quartz crystal. The second and third binding molecules may bring about precipitation of a precipitate on the electrode by any means known in the art. In the present specification, the phrase “capable of bringing about the precipitation of a precipitate” means that the binding molecule has the capability of forming a precipitate. This capability may be intrinsic in the molecule, or may be extrinsic such as, for example, by the binding of one or more additional molecules to the binding molecule, wherein these additional molecules can form the precipitate. An example of such additional molecules is a binding molecule, such as a second antibody (anti-ab), conjugated to an enzyme, such as horseradish peroxidase, glucose oxidase or alkaline phosphatase, which catalyzes a reaction resulting in the precipitation of a reaction product on the electronic transducer.

Chemical compounds to be detected by the method of the invention may be randomly chosen or synthesized, such as by combinatorial chemical methods or from chemical libraries, or chosen using molecular modeling or calculation methods known in the art. In a preferred embodiment, the compounds are screened for their potential use as drugs for reducing or inhibiting the activity of proteases. In a preferred embodiment, the drug inhibits the protease activity either partially or fully by binding to the protease.

It will be understood that this and other aspects of the invention may detect a plurality of chemical compounds at one time, such as in high-throughput screening systems using matricies of chemicals.

In one embodiment, the protease and/or the substrate are translated from an RNA or DNA sample. In this embodiment, the electronic transducer is incubated with the PR substrate after it (and the PR) have been translated from the RNA sample in the presence or in the absence of each of the chemical compounds. In an alternate embodiment, for example when the substrate independently exists, the substrate is first incubated with the electronic transducer and subsequently incubated with the protease in the presence and in the absence of each of the chemical compounds.

In a preferred embodiment, the protease is a viral protease, more preferably a retroviral protease, most preferably an HIV protease. In another preferred embodiment, the chemical compounds are drugs, the substrate is a polyprotein and the at least two segments of the substrate are individual protein components of the polyprotein.

In a second aspect of the invention, there is provided a method for detecting a drug-resistant virus, whose resistance is due to changes in a viral protease, comprising the first aspect of the invention wherein the chemical compounds are anti-viral drugs, the protease is of viral origin, the substrate is a viral encoded polyprotein and the at least two segments of the substrate are individual viral components of the polyprotein.

Examples of anti-viral drugs include the HIV protease inhibitors saquinavir, ritonavir, indinavir and nelfmavir. In a preferred embodiment, an extracted viral RNA sample is translated in the presence or in the absence of an anti-viral drug.

In a third aspect of the invention, there is provided a method for following anti-viral drug resistance in a patient having a viral disease comprising:

(a) obtaining at a first time point a first virus-containing sample from the patient;

(b) analyzing the virus in the first sample for drug resistance according to the method of the invention;

(c) obtaining at a second time point a second virus-containing sample from the patient;

(d) analyzing the virus in the second sample for drug resistance according to the method of the invention; and

(e) comparing the drug resistance determined in steps (ii) and (iv).

In a fourth aspect of the invention there is provided a method for testing a chemical compound for its ability to inhibit the activity of a protease, the protease being capable of specifically cleaving a substrate into at least two segments, the method comprising:

(a) providing a specimen containing a protease, the specimen being divided into a sample A and a sample B;

(b) providing an electronic transducer having bound thereto a first binding molecule capable of specifically binding a first segment of the substrate;

(c) providing a second binding molecule capable of specifically binding a second segment of the substrate, the second binding molecule being capable of bringing about the precipitation of a precipitate on the electronic transducer;

(d) providing a third binding molecule capable of specifically binding the first segment of the substrate at a site other than the site bound by the first binding molecule, the third binding molecule being capable of bringing about the precipitation of a precipitate on the electronic transducer,

(e) incubating the substrate with the sample A in the presence of the chemical compound and with the sample B in the absence of the chemical compound;

(f) dividing the mixture of the substrate with each of samples A and B from step (v) into two sub-samples, being sub-samples A(1) and A(2), and sub-samples B(1) and B(2);

(g) incubating the electronic transducer with each of the sub-samples of step (vi);

(h) incubating the electronic transducer of step (vii) which was incubated with each of sub-samples A(1) and B(1) in the presence of the third binding molecule;

(i) incubating the electronic transducer of step (vii) which was incubated with each of sub-samples A(2) and B(2) in the presence of the second binding molecule;

(j) incubating the electronic transducer of steps (viii) and (ix) under conditions which bring about the precipitation of the precipitate on the electronic transducer; and

(k) measuring a signal from the electronic transducer indicating the presence or absence of the precipitate on the electronic transducer and calculating the ratio of signals obtained, a significant deviation from 1 in the ratio of A(2) to B(2), while the ratio of A(1) to B(1) is approximately 1, indicating that the activity of the protease has been modified by the chemical compound.

In a fifth aspect of the invention there is provided a method for identifying an anti-viral drug capable of reducing or inhibiting the activity of a viral protease, the protease being capable of specifically cleaving a viral polyprotein into at least two individual proteins, the method comprising:

(a) providing a speciman containing viral RNA, the specimen being divided into a sample A and a sample B;

(b) providing an electronic transducer having bound thereto a first binding molecule capable of specifically binding a first protein of the polyprotein;

(c) providing a second binding molecule capable of specifically binding a second protein of the polyprotein, the second binding molecule being capable of bringing about the precipitation of a precipitate on the electronic transducer;

(d) providing a third binding molecule capable of specifically binding the first protein of the polyprotein at a site other than the site bound by the first binding molecule, the third binding molecule being capable of bringing about the precipitation of a precipitate on the electronic transducer,

(e) subjecting the sample A to in vitro translation in the presence of the chemical compound and the sample B to in vitro translation in the absence of the chemical compound;

(f) dividing each of samples A and B from step (v) into two sub-samples, being sub-samples A(1) and A(2), and sub-samples B(1) and B(2), respectively;

(g) incubating the electronic transducer with each of the sub-samples of step (vi);

(h) incubating the electronic transducer of step (vii) which was incubated with each of sub-samples A(1) and B(1) in the presence of the third binding molecule;

(i) incubating the electronic transducer of step (vii) which was incubated with each of sub-samples A(2) and B(2) in the presence of the second binding molecule;

(j) incubating the electronic transducer of steps (viii) and 24(h) under conditions which bring about the precipitation of the precipitate on the electronic transducer; and

(k) measuring a signal from the electronic transducer indicating the presence or absence of the precipitate on the electronic transducer and calculating the ratio of signals obtained, the ratio of A(2) to B(2) being significantly greater than 1, while the ratio of A(1) to B(1) being approximately 1, indicating that the chemical compound may be effective as an anti-viral drug.

In these aspects of the invention, a significant deviation may be considered a deviation of 15% or more from unity.

According to a preferred embodiment of the invention, there is disclosed a rapid assay of retroviral patients, and in particular AIDS patients, for drug resistance. The method is based on measurement of the resistance phenotype of a retroviral protease by the in vitro translation of retroviral mRNA of the virus extracted from the patient in the absence and presence of a retroviral PR inhibiting drug, such as saquinavir, and a comparison of the PR activity under the two different translation paths. A major advantage of the invention is the successful analysis of the respective proteins generated by the minute amounts of mRNA extracted from the blood samples. This success derives from the dual amplification routes involved in the detection scheme: (i) The translation process represents a biocatalytic amplification; (ii) The biocatalyzed precipitation on the electrode represents a biocatalytic amplification that follows a few recognition events on the electrode. Furthermore, the method enables assessing the effectiveness of prescribed drugs, as well as drug doses.

Also provided by the invention are kits for carrying out the methods of the invention. Such kits may include at least the following components: (1) an electronic transducer having bound thereto a first binding molecule capable of specifically binding a first segment of a protease substrate; and (2) a second binding molecule capable of specifically binding a second segment of said substrate, said second binding molecule being capable of bringing about the precipitation of a precipitate on the electronic transducer.

### DETAILED DESCRIPTION OF THE DRAWINGS

In order to understand the invention and to see how it may be carried out in practice, specific embodiments of the invention will now be described, by way of non-limiting example only, with reference to the accompanying drawings, in which:

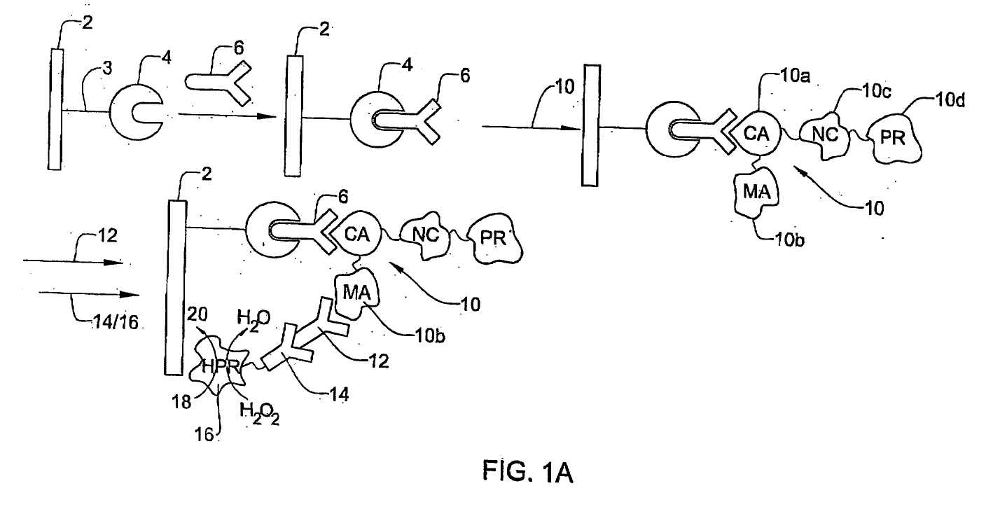

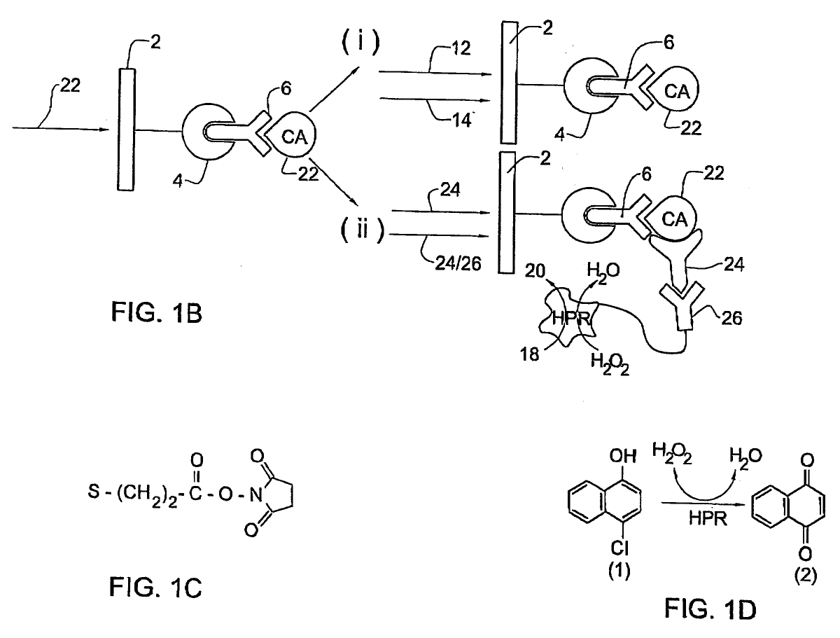

FIG. 1 is a schematic scheme illustrating one embodiment of bioelectronic transduction of translated HIV-1 Gag polyproteins according to the invention:

FIG. 1A—shows bioelectronic transduction in the translated Gag polyproteins obtained in the presence of the PI;

FIG. 1B shows bioelectronic transduction in the translated Gag polyproteins obtained in the absence of PI using anti-MA-Ab (route i) and anti-CA-Ab (route ii);

FIG. 1C shows the linking group used to immobilize protein G to the electrode;

FIG. 1D shows the chemical reaction resulting in the precipitate;

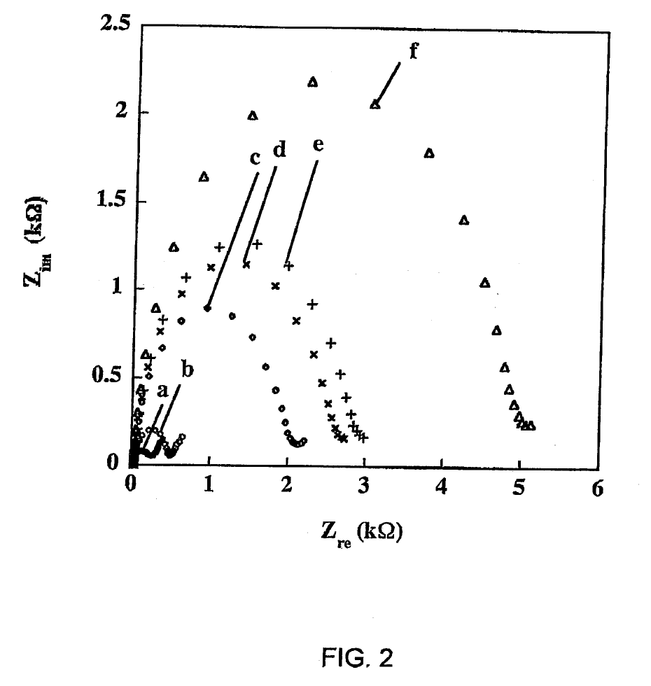

FIG. 2 shows Faradaic impedance spectra (depicted in the form of Nyquist plots, Zᵢₘ vs. Zᵣₑ) that follow the construction of the sensing interface and the analysis of the MA Gag polyprotein translated from bacterial RNA in the presence of the PI, saquinavir, 1×10⁻⁴ M: (a) The G-protein modified Au-electrode. (b) After the immobilization of anti-CA to the surface. (c) After the association of the translated Gag polyprotein. (d) After the binding of anti-MA to the surface. (e) After the association of the anti-MA second antibody/horseradish peroxidase (HRP) conjugate. (f) After the biocatalyzed precipitation of the precipitate in the presence of the HRP substrate, 1×10⁻³ M, and H₂O₂, 1.5×10⁻⁴ M, for a time-interval of 10 minutes. Data were recorded in a 0.1 M phosphate buffer solution, pH=7.0, that includes Fe(CN)₆^{3-/4-}, 1×10⁻² M, as redox label. The electrode was biased at 0.175 V vs. SCE, and an alternating voltage, 10 mV, in the frequency range 100 mHz to 10 kHz was applied;

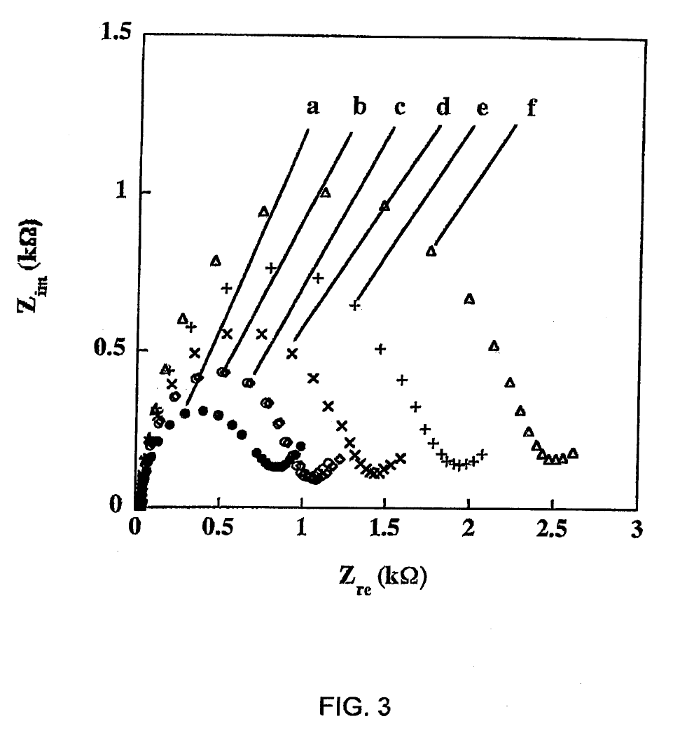

FIG. 3 shows Faradaic impedance spectra (Nyquist plots) corresponding to the analysis of the MA units in the translated Gag polyprotein from bacterial RNA obtained in the absence of the PI: (a) The G-protein modified electrode. (b) After the association of the anti-CA. (c) After binding the translated product. (d) After the association of anti-MA. (e) After the linkage of anti anti-MA/HRP. (f) After the biocatalyzed precipitation of the precipitate in the presence of the HRP substrate, 1×10⁻³ M, and H₂O₂, 1.5×10⁻⁴ M. Experimental details are similar to those detailed in FIG. 2;

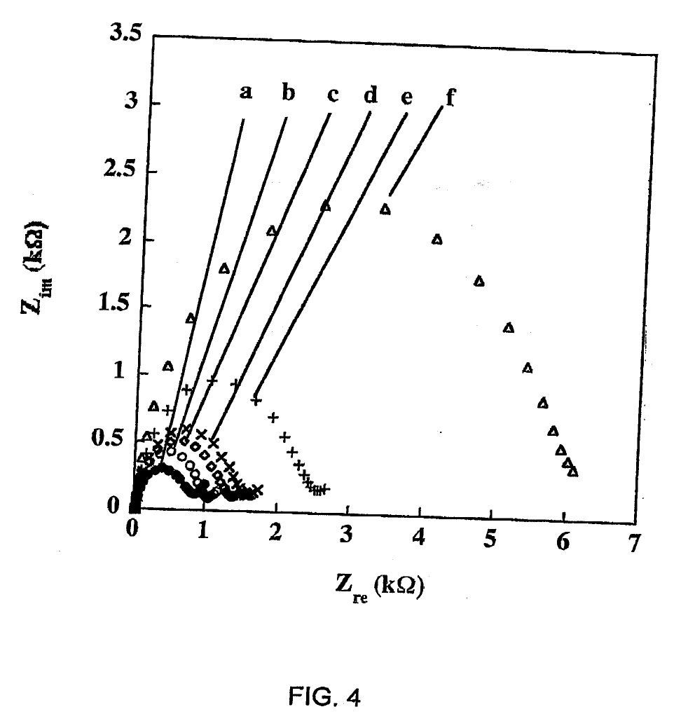

FIG. 4 shows Faradaic impedance spectra (Nyquist plots) corresponding to the analysis of the CA units in the translated proteins obtained from bacterial RNA in the absence of PI: (a) The G-protein modified electrode. (b) After the association of the anti-CA. (c) After binding the translated product. (d) After the association of anti-CA. (e) After binding the anti anti-CA/HRP conjugate. (f) After the biocatalyzed precipitation of the precipitate in the presence of the HRP substrate, 1×10⁻³ M, and H₂O₂, 1.5×10⁻⁴ M. Experimental details as given in FIG. 2;

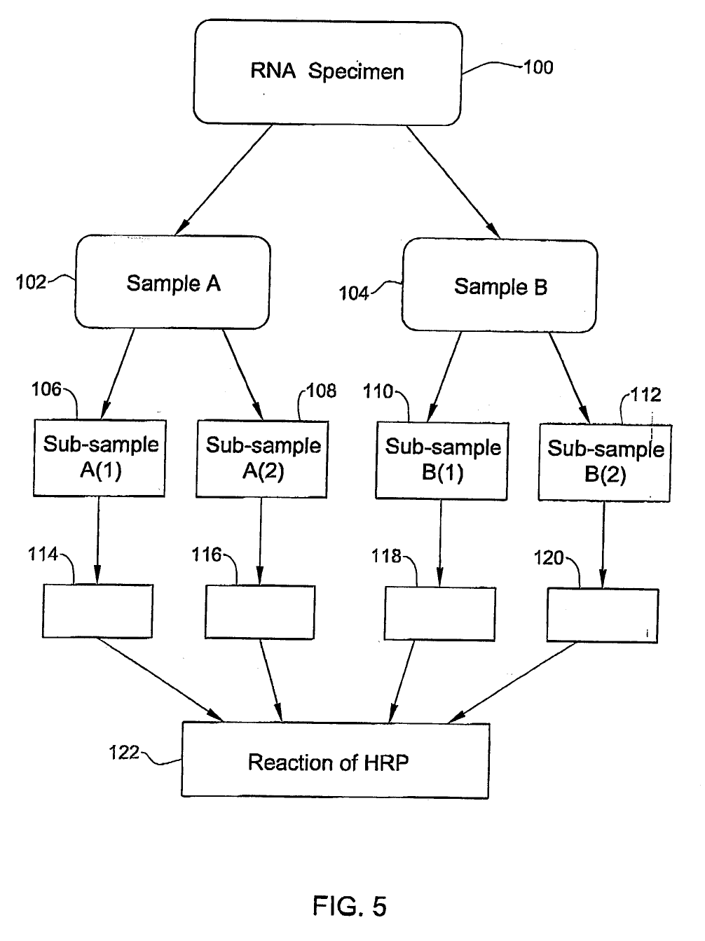

FIG. 5 is a flow diagram illustrating another embodiment of a method according to the invention, being an analytical protocol for the determination of PI resistance in AIDS patients;

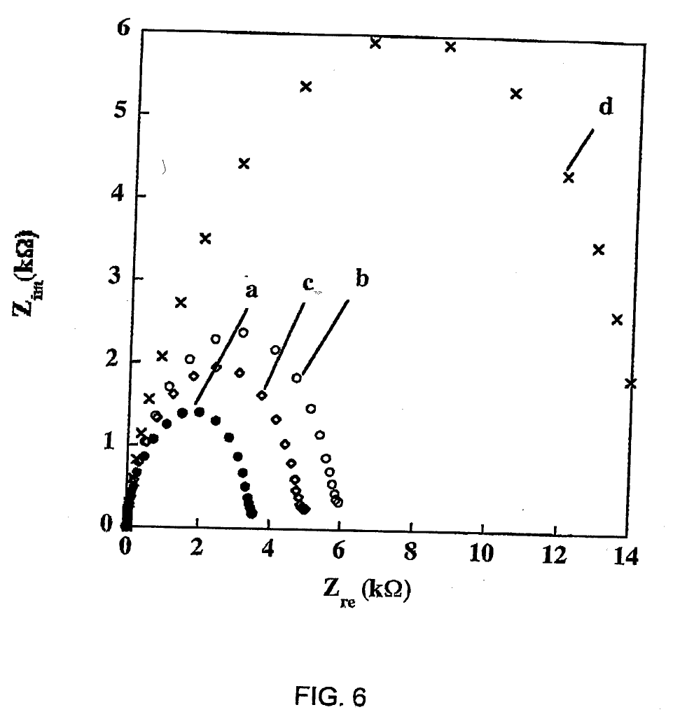

FIG. 6 shows Faradaic impedance spectra corresponding to the analysis of the translated proteins obtained from the mRNA from the media of cultured cells infected by wild-type HIV-1, in the presence and absence of the PI, saquinavir, 1×10⁻⁴ M, according to FIG. 5. Spectra reflect the impedance responses of the electrodes after the final step of the biocatalyzed precipitation of the precipitate in the presence of the HRP substrate, 1×10⁻³ M, and H₂O₂, 1.5×10⁻⁴ M, for a time-interval of 10 minutes: (a) analysis of CA in the absence of PI. (b) Analysis of MA in the absence of PI. (c) The analysis of CA in the presence of PI. (d) Analysis of MA in the presence of PI;

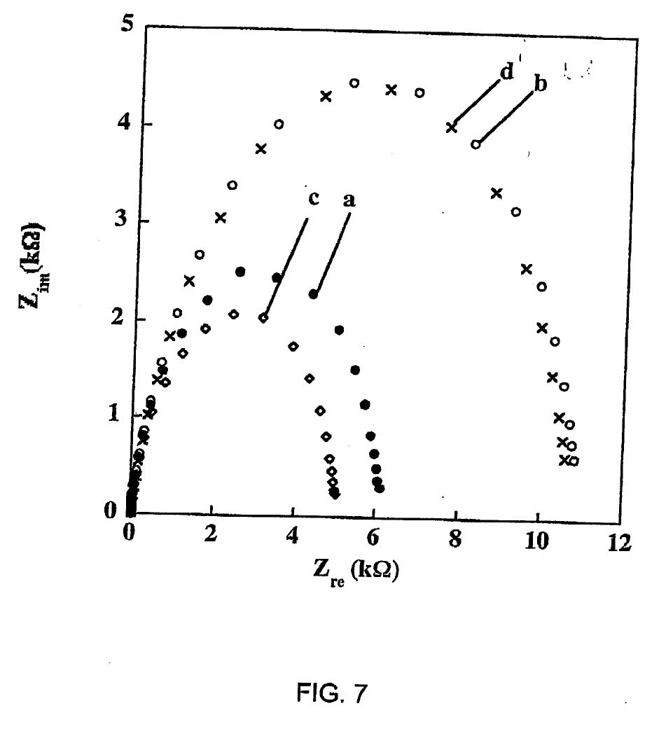

FIG. 7 shows Faradaic impedance spectra corresponding to the analysis of the translated proteins obtained from the mRNA from the media of cultured cells infected with the HIV-1^{G48V} mutant, in the presence and absence of the PI, according to Scheme 2. Spectra depict the impedance responses of the electrodes after the final step of the biocatalyzed precipitation of the precipitate in the presence of the HRP substrate, 1×10⁻³ M, and H₂O₂, 1.5×10⁻⁴ M for 10 minutes: (a) Analysis of CA in the absence of PI. (b) Analysis of MA in the absence of PI. (c) Analysis of CA in the presence of PI. (d) Analysis of MA in the presence of PI; and

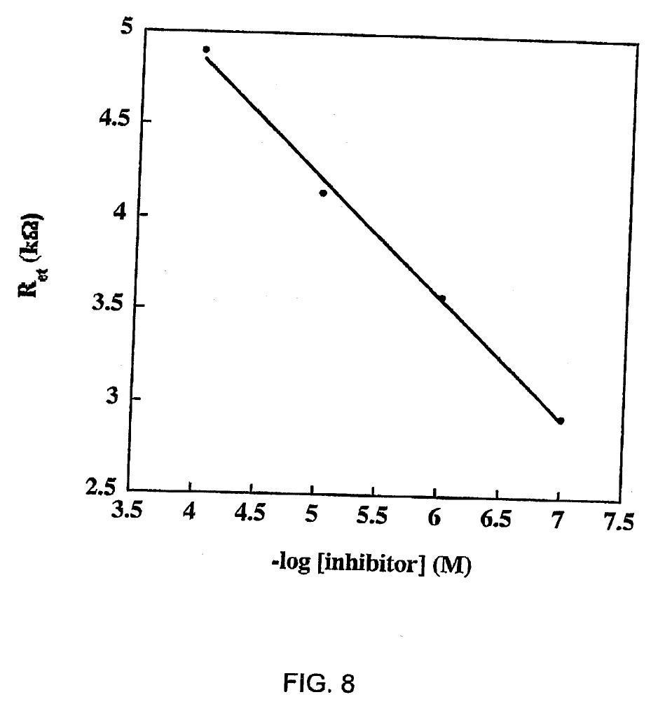

FIG. 8 shows a calibration curve corresponding to the analysis of the MA units in the unprocessed Gag polyprotein obtained from the translation of RNA from cultured cells infected with HIV-1, in the presence of variable concentration of the PI, saquinavir. The calibration curve depicts the observed interfacial electron transfer resistances after the biocatalyzed precipitation of the precipitate on the electrode, upon analysis of the MA units according to FIG. 1A.

### DETAILED DESCRIPTION OF SPECIFIC EMBODIMENTS

Materials & Methods

Chemicals

IgG-goat-anti-rabbit-horseradish peroxidase (HRP), IgG-goat-anti-mouse-HRP, (anti-Ab-HRP), hydrogen peroxide, 4-chloro-1-naphthol, 3.3′-dithiodipropionic acid-bis (N-hydroxysuccinimide) (DSP) active ester, protein G, and the other chemicals were obtained from commercial sources (Aldrich or Sigma) and were used as supplied without further purification. Monoclonal mouse IgG-anti-CA and polyclonal rabbit IgG-anti-MA were prepared by immunization of mice and rabbits with purified recombinant CA and MA proteins, respectively. The biotin in vitro translation kit was purchased from Roche Diagnostics, Mannheim, Germany. The PR inhibitor saquinavir is commercially available, and was initially dissolved in 10% dimethyl sulfoxide (DMSO) to a concentration of 1.0 mM and stored at −20° C. until further use. Ultrapure water from Elgastat (VHQ) source was used throughout the experiments.

Characterization and Pretreatment of Electrodes

Gold wire electrodes (0.5 mm diameter, ˜0.2 cm² geometrical area, roughness coefficient ˜1.2-1.5) were used for the electrochemical measurements. To remove any previous organic layer, and to regenerate a bare metal surface, the electrodes were treated in a boiling 2M solution of KOH for 4 h, then rinsed with water, and stored in concentrated sulfuric acid. Prior to modification, the electrodes were rinsed with water, dried, and soaked for 2 min in fresh piranha solution (30% H₂O₂, 70% H₂SO₄). The resulting electrodes were then rinsed with water, soaked for 10 mmn in concentrated nitric acid, and again rinsed with water.

Electrochemical Measurements

A conventional three electrode cell, consisting of the modified Au electrode, a glassy carbon auxiliary electrode isolated by a glass frit, and a saturated calomel electrode (SCE) connected to the working volume with a Luggin capillary, was used for the electrochemical measurements. The cell was positioned in a grounded Faradaic cage. Impedance measurements were performed using an electrochemical impedance analyzer (EG&G, model 1025) and potentiostat (EG&G, model 283) connected to a computer (EG&G Software Power Suite 1.03 and 270 for impedance measurements). All electrochemical measurements were performed in 0.1 M phosphate buffer, pH 7.0, as a background electrolyte solution. Faradaic impedance measurements were performed in the presence of 10 mM K₃[Fe(CN)₆]/K₄Fe(CN)₆] (1:1) mixture, as a redox probe. Impedance measurements were performed at a bias potential of 0.17 V versus SCE using alternating voltage, 10 mV, in the frequency range of 100 mHz to 10 kHz. The impedance spectra were plotted in the form of complex plane diagrams (Nyquist plots).

Microgravimetric Measurements

A QCM analyzer (Fluke 164T multifunction counter, 1.3 GHz, TCXO) linked to a personal computer and a homemade flow cell with a working volume of 0.3 mL was employed. Quartz crystals (AT-cut, 9 MHz, EG&G) sandwiched between two Au electrodes (area 0.196 cm², roughness factor ˜3.5) were used. The Au-quartz crystals were cleaned by a piranha solution (30% H₂O₂, 70% H₂SO₄) followed by rinsing with water.

Electrode Modifications

The electrodes were rinsed with water, dried, and soaked in a 10 mM solution of the DSP-active ester in DMSO for 30 min at room temperature. The functionalized electrodes were rinsed with DMSO and water and incubated in a 100 μg·m]L⁻¹ solution of protein G, for 90 min in a phosphate buffer saline solution, pH=7.4, at room temperature, to couple covalently protein G lysine residues to the functionalized electrodes. The protein G-functionalized electrodes were then allowed to interact with the Fc fragment of the anti-CA-antibody, 1 μg·mL⁻¹, 30 min at room temperature, to yield the sensing interfaces.

Analytical Procedure

The antibody-functionalized electrode was interacted with varying concentrations of the Gag polyprotein generated in the translation mixture for 60 min. diluted to 1.0 mL in phosphate-buffered saline (PBS) solution, 0.1 M, pH 7.0, at room temperature. After attachment of the respective polyprotein to the sensing interface, the electrode was incubated in the solution of anti-MA or anti-CA (sources were 200-fold diluted) for 30 min at room temperature. After attachment to the respective antibody, the electrode was incubated either in a goat-anti-rabbit-HRP conjugate solution (2.5 μg·mL⁻¹), for the MA analysis, or in a goat-anti-mouse-HRP conjugate solution (2.5 μg·mL⁻¹) for the CA analysis, for 30 minutes at room temperature. 4-Chloro-1-naphthol (1) (FIG. 1D) was dissolved initially in ethanol and then the ethanolic stock solution was diluted with 0.1 M phosphate buffer, pH 7.0, to yield the developing solution that included 1×10⁻³ M (1) and 2% (v/v) ethanol. The modified electrodes consisting of the HRP-tagged antibody-functionalized electrodes were incubated in the developing solution of (1) for a fixed and controlled time of 10 minutes at room temperature to stimulate the precipitation of (2) (FIG. 1D).

After incubation of the respective electrodes in the probe solution, the electrodes were rinsed with 0.1 M phosphate buffer, pH 7.0, and introduced into the electrochemical cell for their analyses by Faradaic impedance spectroscopy. It should be noted that after each step the electrode was rinsed thoroughly with buffer solution, pH 7.0, to eliminate any non-specific adsorbates on the electrode. In the microgravimetric quartz crystal microbalance measurements, the various modification and amplification steps, including the rinsing steps, were performed in the flow cell of the QCM apparatus.

Cells and Viruses

Sup T1 cells were maintained in RPM1 1640 medium supplemented with 10% fetal calf serum, antibiotics (penicillin and streptomycin) and 2 mM glutamine. HIV-1_{IIIB} infect (kindly supplied by Dr. Wainberg, Lady Davis Institute, Montreal, Canada) was used to infect the cultured cells at 0.1 multiplicity of inspection, (MOI), and 7-9 days post-infection virus was harvested. Culture medium containing the virus was clarified from cell debris by centrifugation at 10,000 rpm for 10 minutes and the clear supernatant was centrifuged for 45 min at 45,000 rpm in a Beckman centrifuge (SW 50.1 rotor). The drug resistant HIV-1 (NL4-3) strain that includes the G48V mutated PR was constructed and propagated as previously described (Blumenzweig, I. et al. HIV-1 Vif-derived peptide inhibits drug-resistant HIV proteases. Biochem. Biophys. Res. Commun. 292, 832-840 (2002).

Blood Samples

Plasma was obtained from consenting donors following removal of blood cells by centrifugation for 5 minutes at 3,000 rpm. The plasma was 3-fold diluted with PBS, and viral particles were peletted by centrifuging the volume for 45 minutes at 45,000 rpm in a Backman centrifuge (SW 50.1 rotor). The viral pellets were suspended in 1 mL TRIzol reagent (Gibco BRL) containing 5-10 μg of tRNA (Sigma) as carrier and RNA was extracted according to the manufacturer's instructions.

In Vitro Translation

All the RNA preparations were translated in vitro using the “Biotin in vitro translation kit” (Roche 1559951) in the absence or presence of Saquinavir (Ro 31-8959). Reactions were performed in 50 μL, according to the manufacturer's instructions, using DEPC-treated water. The PR inhibitor Saquinavir was dissolved in 1M NaCl in the DEPC-treated water, and was added to reaction mixtures before starting the synthesis. The reaction mixtures were incubated for 90 min at 30° C. and the reactions were halted by placing in ice or storage at −75° C. until further use in the electrochemical measurements.

### EXAMPLES

### Example I

An RNA extract obtained from HIV patients' blood samples may be translated in vitro. This results in the synthesis of the viral Gag polyprotein, an integral part of which is the viral protease (PR). The protease autohydrolyzes (processes) the polyprotein precursor to yield active virions. The bioelectronic method described below allows comparison of the processing efficiency of the PR by comparing the amounts of cleaved and non-cleaved viral precursors by the intrinsic protease.

The scheme of FIG. 1A exemplifies one part of the method of the invention for probing HIV drug resistance through analysis of the protease inhibition. A protein G molecule (4) is immobilized on an electronic transducer such as an Au-electrode (2) via a linker molecule (3). A non-limiting example of a linker molecule is shown in FIG. 1C. The protein G molecule (4) binds an anti-CA antibody (6), CA being one of the individual protein segments of the viral Gag polyprotein (10). In vitro translation of an RNA extract from HIV patients' plasma in the presence of a protease inhibitor (PI) such as saquinavir results in translation of the uncleaved viral Gag polyprotein (10), which comprises the viral capsid protein segments CA (10a), MA (10b) and NC (10c), as well as the viral protease PR (10d). The Gag polyprotein (10) is not cleaved because the protease activity is inhibited by the PI. The immobilized anti-CA (6) binds Gag polyprotein (10) through the CA protein segment (10a). This results in the binding of the uncleaved Gag proteins MA (10b)-CA (10a)-NC (10c) through the anti-CA antibody (6), and protein G (4) and the linker (3) to the electrode (2).

Incubation of the electrode-bound Gag polyprotein (10) with an anti-MA antibody (12) results in the anti-MA antibody (12) binding to the MA protein segment (10b). Subsequently, the electrode (2) is incubated with an anti-anti-MA second antibody (14) conjugated to an HRP molecule (16) resulting in the binding of the anti-anti-MA antibody (14) to the uncleaved Gag polyprotein through the anti-MA antibody (12). This is followed by the biocatalyzed H₂O₂-mediated oxidation of 4-chloronaphthol (18) to the precipitate (20), as further illustrated in FIG. 1D. The PR inhibition is thus assayed by the biocatalyzed precipitation of the precipitate (20) on the electrode (2) or on an Au-quartz crystal using Faradaic impedance spectroscopy or microgravimetric quartz-crystal microbalance measurements, respectively. This amplifies the primary recognition of the non-cleaved Gag precursor assembly (10).

Note that the extent of precipitation is controlled by the inhibition efficiency of the PI, and as the inhibitory effect decreases, the accumulation of precipitate (20) on the transducers (2) is reduced. It should be noted that the detection of the non-hydrolyzed Gag polyprotein (10) is a consequence of two amplification steps: In the first step the translation of the viral RNA to the protein provides an amplification path. The second amplification step involves the biocatalyzed precipitation of (20) on the transducers (2). As a single recognition event of the non-hydrolyzed polyprotein (10) by the anti-CA (6) sensing interface is translated into the accumulation of many insoluble molecules on the transducer (2), the precipitation process presents an effective amplification route. The formation of the precipitate on the electronic transducer may be probed by electrochemical means (Faradaic impedance spectroscopy) or by microgravimetric quartz crystal microbalance measurements.

In FIG. 1B, on the other hand, in vitro translation of the RNA extract occurs in the absence of the PI, resulting in translation of a viral Gag polyprotein which is cleaved by a filly functional protease. This results in the release of the individual protein segments, the CA segment (22) being bound to the electrode (2) by the immobilized anti-CA antibody (6). In route (i), incubation with the anti-MA antibody (12) and the anti-anti-MA second antibody-HRP conjugate (14) does not result in binding to the CA (22). Thus the biocatalyzed precipitation of the precipitate (20) onto the electrode (2) is retarded in route (i) (provided non-specific adsorption processes are minimized).

To confirm the formation of the anti-CA antibody (6)/CA (22) complex in the system that includes the functional PR, the resulting interface is reacted with a second anti-CA antibody (24) in FIG. 1B, route (ii). This second anti-CA antibody (24) binds CA at a site other than the site bound by the immobilized anti-CA antibody (6). The anti-Ab-HRP conjugate (26) is incubated with the interface and binds to the second anti-CA antibody (24), thus becoming linked to the electrode (2) surface. Then the reaction of biocatalyzed H₂O₂-mediated oxidation of 4-chloronaphthol (18) occurs, and yields the precipitate (20), which precipitates on the electrode.

### Example II

The development of the bioelectronic schemes for the analysis of HIV-1 drug resistance was carried out in the following phases: (i) Assessment of viral RNA extracted from bacteria expressing viral proteins in the presence and absence of the PR inhibitor. (ii) The assessment of viral RNA extracted from cells infected by wild-type HIV-1 or by a drug-resistance HIV-1 mutant in the absence or presence of the PR inhibitor. (iii) The assessment of RNA extracted from blood samples of AIDS patients by the synthesis of the viral proteins in the absence or presence of the PR-inhibitor. The method enables us to assess the relative amounts of drug-resistant PR, as well as relative concentrations of inhibitors required to inhibit PR.

Impedance spectroscopy is an effective method for probing the features of surface-modified electrodes. The complex impedance can be presented as the sum of the real, Zᵣₑ(ω), and imaginary, Zᵢₘ(ω), components that originate mainly from the resistance and capacitance of the electrode interface, respectively. Modification of the metallic surface with a biomaterial or an organic layer decreases the double-layer capacitance and retards the interfacial electron-transfer kinetics. The electron-transfer resistance at the electrode is given by eq. 1, where R_{Au} and R_{mod} are the electron-transfer resistance of the unmodified electrode and the variable electron-transfer resistance introduced by the modifier, in the presence of the solubilized redox probe, respectively. A typical shape of a Faradaic impedance spectrum (presented in the form of a Nyquist plot, Zᵢₘ versus Zᵣₑ at variable frequencies) includes a semicircle region lying on the Zᵣₑ axis followed by a straight line. The semicircle portion, observed by higher frequencies, corresponds to the electron-transfer-limited process, whereas the linear part is characteristic of the lower frequencies range, and represents the diffusional-limited electron-transfer process. The diameter of the semicircle corresponds to the electron-transfer resistance at the electrode surface, Rₑₜ.

Rₑₜ=R_{Au}+R_{mod} (1)

The precipitation of the insoluble product on the electrode support can also be probed by microgravimetric quartz crystal microbalance QCM analyses. The analysis of the Gag polyproteins according to FIGS. 1A and 1B involves the binding of the proteins to the sensing interface, followed by the association of the detecting antibody and the anti-Ab/enzyme conjugate. These binding processes alter the mass on the piezoelectric crystal. The subsequent biocatalyzed precipitation of the precipitate on the crystal represents a time-dependent mass change occurring on the transducer. The frequency change of a quartz crystal, Δf, resulting from a mass alteration on the crystal, Δm. is given by the Sauerbrey relation, eq. 2, where fₒ is the resonance frequency of the quartz crystal, A is the piezoelectrically active area, p_{q} is the density of quartz (2.648 g·cm⁻³), and μ_{q} is the shear modulus (2.947×10¹¹ dyn·cm⁻² for AT-cut quartz).

$$Δ f = - 2 {f}_{o}^{2} \frac{Δ m}{{A ( {μ}_{q} \cdot {ρ}_{q} )}^{1 / 2}} \qquad ( 2 )$$

FIG. 2 shows the Faradaic impedance spectra that correspond to the build-up of the sensing interface and to the analysis of the in vitro translation of the total bacterial RNA in the presence of saquinavir, according to FIG. 1A. The redox-label in the electrolyte solution is Fe(CN)₆³⁻/Fe(CN)₆⁴⁻. The electron transfer barrier (resistance) to the redox-label, as a result of the formation of the protein layers and the biocatalytic generation of the insoluble product on the electrode surface, are employed to probe the translation process. The stepwise association of protein G and the anti-CA antibody results in an increase in the electron-transfer resistances at the electrode surface to 200 and 400 Ω (curves a and b), respectively. This increase in the interfacial electron-transfer resistances is attributed to the partial hydrophobic insulation of the electrode support by the proteins. Parallel microgravimetric QCM analyses indicate that the surface coverage of protein G is ca. 8.8×10⁻¹¹ mol·cm⁻² and of anti-CA-Ab ca. 2.0×10⁻¹² mol·cm-⁻².

Binding of the Gag polyprotein (10) through CA (10a) to the anti-CA-Ab (6) further increases the interfacial electron-transfer resistance to 2100 Ω (curve c). The association of the anti-MA-Ab (12) to the interface increases the interfacial electron-transfer resistance to Rₑₜ=2800 Ω (FIG. 2, curve d). The increase in the interfacial electron transfer resistances upon the association of the Gag polyprotein and anti-MA-Ab is consistent with the fact that the association of the proteins insulates the electrode surface and perturbs the interfacial electron transfer to the redox-label solubilized in the electrolyte solution. Upon addition of the anti-anti-MA-HRP conjugate there is a further increase in the electron transfer resistance to Rₑₜ=3000 Ω (curve e). The subsequent biocatalyzed precipitation of (20) results in a significant increase in the interfacial electron transfer resistance, Rₑₜ=5000 0 (curve f), indicating that an amplified detection of the Gag poplyprotein is indeed observed, and implying that the inhibition of the protease in the Gag precursor did occur.

FIG. 3 shows the Faradaic impedance spectra obtained upon the conduction of the in vitro translation of the Gag polyprotein in the absence of the PR inhibitor. This experiment reflects the protease activity according to FIG. 1B, route (i). Curve a shows the spectrum observed upon the attachment of protein G to the surface of an Au-electrode; curve b depicts the spectrum obtained upon the attachment of the Fc fragment of the anti-CA antibody to the Protein G on the electrode support, and curve c shows the spectrum after the attachment of the translated CA units obtained under conditions where proteolysis of the Gag precursor occurred. Curve d corresponds to the spectrum obtained after an attempt to bind the anti-MA antibody to the sensing interface, and curves e and f correspond to the subsequent attempts to bind the anti-anti-MA-HRP conjugate, and to stimulate the biocatalyzed precipitation of the precipitate (20), respectively.

Interestingly, we find that upon the treatment of the system with the anti-MA antibody, an increase in the interfacial electron transfer resistance, ΔRₑₜ≈400 Ω is observed, implying that the antibody binds to the sensing interface, even though the MA sites should not exist on the surface due to the proteolytic activity of PR. The associated anti-MA-Ab to the interface stimulates the binding of the anti-Ab-HRP conjugate and the precipitation of the insoluble product. However, the translation of identical quantities of mRNA in the absence and presence of the Saquinavir inhibitor may yield substantially different electron transfer resistances: While the inhibited processing yields an electrode with a high electron transfer resistance, ca. 5100 Ω and an increase in the interfacial electron transfer resistance of ΔRₑₜ≈2000 Ω upon the precipitation of (20), the electrode treated with the non-inhibited translation mixture yields an electrode with an interfacial electron transfer resistance of only 2500 Ω, and an increase in the interfacial electron resistance of ΔRₑₜ=500 Ω upon the precipitation of (20), a value that is 4-fold lower than the value observed for the inhibited translation mixture. The binding of the anti-MA-Ab to the sensing interface and the subsequent precipitation of (20) under conditions where the translation of the mRNA is performed without inhibition, is attributed to the existence of unprocessed polyprotein that was not cleaved by the PR. This unprocessed polyprotein acts as a background perturbation for the analysis of the non-cleaved polyprotein generated upon translation in the presence of the PI. Thus, for practical analysis of HIV-drug resistance it is mandatory to develop an analysis that assays, in parallel, the background level of unprocessed protein, and the polyprotein content generated by translation in the presence of the PI.

Albeit the sensing interface includes a low coverage of the unprocessed polyprotein, it also includes a high-content of the hydrolyzed CA units, generated in the translation mixture. This was confirmed by the analysis of the modified electrode according to FIG. 1B, route (ii) and is depicted in FIG. 4. In this experiment the sensing interface consisting of the anti-CA antibody is treated with the translation mixture to fish out the hydrolyzed CA and residual unprocessed polyprotein, curve (a). The increase in the interfacial electron-transfer resistance to Rₑₜ=1600 Ω indicates the binding of proteins to the surface. Further association of a second anti-CA-Ab and the anti-anti-CA-Ab/HRP conjugate, curves (d) and (e), respectively, followed by the precipitation of (20) leads to a pronounced increase in the interfacial electron transfer resistance of the electrode to Rₑₜ=2000 Ω. The increase in the interfacial electron transfer resistance as a result of the precipitation of (20) is ΔRₑₜ≈3600Ω. This difference is higher than the value observed upon the precipitation of (20) in the presence of the anti-MA-Ab (Cf. FIG. 2, ΔRₑₜ=2000 Ω) and is attributed to the higher affinity of the anti-CA-Ab as compared to the anti-MA-Ab to the respective antigens.

Microgravimetric quartz-crystal-microbalance experiments further confirm the results and conclusions extracted from the Faradaic impedance measurements. Table 1 summarizes the frequency changes of functionalized Au-quartz crystals upon the analysis of the MA in the translated Gag polyprotein in the presence and absence of the PR inhibitor, saquinavir, and the analysis of the CA in the translated polyprotein in the absence of the inhibitor. In these experiments total RNA from cultured cells was employed. In all of the systems, the sensing interface consists of the protein G as base monolayer, 8.8×10⁻¹¹ mole-cm⁻², and the associated anti-CA layer as the recognition interface.

Entry (a) of Table 1 summarizes the frequency changes observed upon the stepwise analysis of the Gag polyprotein translated in the presence of the inhibitor, according to FIG. 1A. The frequency changes, −50 Hz, observed upon treatment of the surface with the translated proteins, and upon interaction with the anti-MA, −80 Hz, indicate that an unprocessed Gag polyprotein is generated upon translation. The biocatalyzed precipitation of (20) results in a frequency change of Δf=−100 Hz, consistent with the effective formation of a precipitate on the transducer. Entry (b) summarizes the frequency changes observed upon the analysis of the non-processed Gag polyprotein in the translation mixture obtained in the absence of the inhibitor. The frequency change, −40 Hz, observed upon the precipitation of (20) implies that non-cleaved Gag polyprotein exists in the translation mixture, consistent with the conclusion obtained from the impedance measurements.

Table 1, entry (c), summarizes the frequency changes upon the analysis of the CA unit obtained upon translation in the absence of the inhibitor, according to FIG. 1B, route (ii). In this case, the cleaved, as well as non-cleaved, CA are analyzed. The biocatalyzed precipitation of (20) results in a frequency change of −150 Hz, indicating that anti anti-CA was associated to the sensing interface. Note that the formation of (20) in the presence of anti-CA is enhanced as compared to the effectiveness of the generation of (20) in the presence of the anti-MA. This observation is in agreement with the Faradaic impedance analyses, and may be attributed to the higher affinity of the anti-CA to the respective precursor.

TABLE 1
Microgravimetric quartz-crystal-microbalance
analyses of the processed MA and CA units in the
presence and absence of the Saquinavir PR inhibitor.^{a}

| | | | | | Precip- |
|---|---|---|---|---|---|
| | Anti- | | Anti-anti | Anti anti- | itation |
| | MA | Anti-CA | MA/HRP | CA/HRP | of (2) |
| | Δf (Hz) | Δf (Hz) | Δf (Hz) | Δf (Hz) | Δf (Hz) |
| (a) Translated | −80 | — | −140 | — | −100 |
| Gag polyprotein | | | | | |
| in the presence of | | | | | |
| PR inhibitor | | | | | |
| (b) Translated | −15 | — | −30 | — | −40 |
| Gag polyprotein | | | | | |
| in the absence of | | | | | |
| PR inhibitor | | | | | |
| (c) Translated | — | −150 | — | −110 | −150 |
| proteins in the | | | | | |
| absence of PR | | | | | |
| inhibitor | | | | | |

^{a}Frequency changes are determined at time-intervals identical to the impedance measurements.

### Example III

The experiments described above demonstrate that it is possible to electronically transduce the differences in the translation of mRNA in the presence and absence of the PI. A further, more precise assay protocol is outlined in FIG. 5.

An extracted RNA specimen 100 is subdivided into two samples, A 102 and B 104. While sample A is subjected to in vitro translation in the presence of the PI, sample B is translated in the absence of the PI. Each of the translation mixtures is then further divided into two equal sub-samples, sample A into sub-samples A(1) 106 and A(2) 108, and sample B into sub-samples B(1) 110 and B(2) 112. The sub-samples A(1) and B(1) are subjected to the analysis of the total CA units using anti-CA, according to FIG. 1B, route (ii). The sub-samples A(2) and B(2) utilize the anti-MA antibody to assess content of polyprotein in the respective samples, according to FIG. 1A and FIG. 1B, route (i). Note that the ratio of the interfacial electron transfer resistances generated upon the precipitation of the precipitate (20) in sub-samples A(2) and B(2) reflect the content of polyprotein generated under PR inhibition vs. the unprocessed (=unhydrolyzed) polyprotein in the system.

The ratio of electron transfer resistances of paths A(1) and B(1) should be independent of the PI, and its value should be ≈1. This ratio provides an internal standard for the effectiveness of translations in the entire set of experiments. Thus, the four-path analysis scheme of the mRNA translation processes eliminates the background signal of unprocessed polyproteins in the absence of the inhibitor. The assay also abrogates the difference in the affinities of anti-CA and anti-MA to the respective antigens. Since the original sample is subdivided into equivalent sub-samples prior to translations, the problem of different RNA contents in analysis paths is cancelled out.

To verify the analysis method shown in FIG. 5, we applied it to analyze the translation processes of the wild type HIV-1 in the absence and presence of the PI, and in parallel to analyze the translation processes of RNA extracted from the G48V mutated virus which is resistant to saquinavir. FIGS. 6 and 7 show the Faradaic impedance spectra corresponding to the final step of precipitation of 20 on the electrode supports after the application of the analytical protocol outlined in FIG. 5.

FIG. 6 shows the spectra obtained for the translation of the mRNA derived from an RNA extract from the media of cultured cells infected by the wild type virus. Curves (a) and (b) of FIG. 6 show the spectra in the absence of the PI (sample B), according to paths B(1) (110) and B(2) (112) in FIG. 5, respectively. Curves (c) and (d) of FIG. 6 are the Faradaic impedance spectra observed for the Au-electrodes obtained in the presence of saquinavir as the PI (sample A), according to paths A(1) and A(2) in FIG. 5, respectively. As expected, the electron transfer resistance for the inhibited virus analyzed with anti-MA for the polyprotein according to path A(2) (curve d) is the highest and corresponds to Rₑₜ=14 kω. For comparison, the use of the anti-MA for analyzing the non-inhibited protease encoded by the wild-type viral RNA according to path B(2) (curve b) yields an electron transfer resistance of Rₑₜ=6 kω. Clearly, the ratio of electron transfer resistances of the inhibited vs. non-inhibited PR is Rₑₜ^{B(2)}/Rₑₜ^{A(2)}=0.43.

An identical analysis was applied to analyze the mutated HIV-1^{G48V}, which is resistant to saquinavir. Curves (b) and (d) of FIG. 7 show the parallel analyses of the translation processes of RNA extracted from a G48V mutant according to paths B(2), (without inhibitor, sample B), and A(2) (with the inhibitor, sample A), respectively. The resulting interfacial resistances are identical in the two systems, Rₑₜ≈11 kΩ, indicating that no effect of the inhibitor is observed on the protease activity. The ratio Rₑₜ^{B(2)}/Rₑₜ^{A(2)}=1 is observed for the G48V system, as expected. Note, however, that there is a difference in the electron transfer resistances resulting upon analyzing the wild-type virus according to path B(2) (FIG. 6, curve b) (Rₑₜ=6 kΩ) as compared to the mutated HIV-1^{G48V} analyzed by path B(2) (FIG. 7, curve b) (Rₑₜ=11 kΩ). This implies that the protease-activity of the wild-type virus and of the HIV-1^{G48V} differ, and that the PR of the mutant has a lower activity for processing (hydrolyzing) the polyprotein.

### Example IV

The method outlined in the present study enables the development of a quantitative assay to probe the effect of the inhibitor concentration on the activity of the viral PR. Since the interfacial electron transfer resistance analyzed according to FIG. 1(A) directly translates to the content of non-cleaved Gag polyprotein, the interfacial electron transfer resistance correlates with the inhibition efficiency of the translated PR activity by the specific inhibitor. FIG. 8 shows the calibration curve that corresponds to the interfacial electron transfer resistances of electrodes that analyze the Gag polyproteins translated from the RNA of HIV-1 infected cultured cells, in the presence of increased concentrations of the saquinavir inhibitor. Clearly, as the concentration of the saquinavir inhibitor increases, the interfacial electron transfer resistance is enhanced, consistent with the elevated content of non-cleaved Gag polyprotein. Such quantitative correlation between the inhibitor concentration and the content of non-cleaved polyprotein may be of extreme importance in determining drug doses and drug efficiencies.

### Example V

The analysis scheme outlined in FIG. 5 was applied to detect drug resistance in AIDS patients, treated with saquinavir. In this process, we expect that the analysis of the CA-units by the anti-CA antibodies will lead to an electron transfer resistance ratio of paths A(1) 106 and B(1) 110 in FIG. 5, that corresponds to α=Rₑₜ^{B(1)}/Rₑₜ^{A(1)}≈1.0 whereas the ratio of electron transfer resistance analyzing the MA-units with the anti-MA antibody of paths B(2) 112 and A(2) 108 in FIG. 5 should reveal the value β=Rₑₜ^{B(2)}/Rₑₜ^{A(2)}≈1.0 for drug resistant patients, and β<1 for drug sensitive patients.

Table 2 summarizes the results observed for five patients. From these values it may be predicted that patients #2 and #5 will develop drug resistance. It is difficult to verify the validity of these conclusions, and it is necessary to rely on the subjective statements of the physicians who treated the patients. Based on this information, patients #2 and #5 were, indeed, under severe conditions which did not react to the treatment with saquinavir, whereas all other patients were under balanced conditions. Furthermore, the results depicted in Table 1 suggest that patient #1 may develop partial resistance to the drug.

TABLE 2
Analysis of HIV Patients for Saquinavir Drug Resistance^{a}

| Patient # | α = Rₑₜ^{B(1)}/Rₑₜ^{A(1)} | β = Rₑₜ^{B(2)}/Rₑₜ^{A(2)} |
|---|---|---|
| 1 | 1.17 ± 0.03 | 0.87 ± 0.04 |
| 2 | 1.07 ± 0.03 | 1.1 ± 0.04 |
| 3 | 1.04 ± 0.03 | 0.75 ± 0.04 |
| 4 | 1.10 ± 0.03 | 0.79 ± 0.04 |
| 5 | 1.0 ± 0.03 | 1.07 ± 0.04 |

^{a}Analyzed according to the scheme of FIG. 5.

In the method claims that follow, alphabetic characters and Roman numerals used to designate claim steps are provided for convenience only and do not imply any particular order of performing the steps.

1. A method for detecting whether a tested chemical compound has the ability to inhibit the activity of a protease, said protease being capable of specifically cleaving a substrate into at least two segments, the method comprising:
(a) providing an electronic transducer having bound thereto a first binding molecule capable of specifically binding a first segment of said substrate;
(b) providing a second binding molecule capable of specifically binding a second segment of said substrate, said second binding molecule being capable of bringing about the precipitation of a precipitate on said electronic transducer;
(c)incubating said substrate with said protease in the presence and in the absence of said chemical compound;
(d) incubating the electronic transducer with the substrate of step (c);
(e)incubating the electronic transducer of step (d) in the presence of said second binding molecule;
(f) incubating the electronic transducer of step (e) under conditions which bring about the precipitation of the precipitate on said electronic transducer; and
(g) detecting the presence or absence of the precipitate on said electronic transducer, a difference in the amount of precipitate in the presence and in the absence of said chemical compound indicating inhibition of the activity of the protease by the compound.

2. A method according to claim 1 further comprising providing a third binding molecule capable of specifically binding the first segment of said substrate at a site other than the site bound by the first binding molecule, said third binding molecule being capable of bringing about the precipitation of a precipitate on said electronic transducer, incubating the electronic transducer of step (iv) in the presence of said third binding molecule and detecting the presence or absence of the precipitate on said electronic transducer, the presence of the precipitate indicating that the first binding molecule has bound the first segment.

3. A method for detecting whether a tested chemical compound has the ability to inhibit the activity of a protease, said protease being capable of specifically cleaving a substrate into at least two segments, the method comprising:
(a) providing an electronic transducer having bound thereto a first binding molecule capable of specifically binding a first segment of said substrate;
(b) providing a second binding molecule capable of specifically binding a second segment of said substrate, said second binding molecule being capable of bringing about the precipitation of a precipitate on said electronic transducer;
(c)incubating the electronic transducer with said substrate;
(d) incubating the electronic transducer of step (c) with said protease in the presence and in the absence of each of said chemical compounds;
(e)incubating the electronic transducer of step 1(d) in the presence of said second binding molecule;
(f) incubating the electronic transducer of step 1(e) under conditions which bring about the precipitation of the precipitate on said electronic transducer; and
(g) detecting the presence or absence of the precipitate on said electronic transducer, a difference in the amount of precipitate in the presence and in the absence of said chemical compound indicating inhibition of the activity of the protease by the compound.

4. A method according to claim 1 wherein said first and second binding molecules are antibodies.

5. A method according to claim 1 wherein said substrate is a polyprotein.

6. A method according to claim 5 wherein said at least two segments are individual protein molecules.

7. A method according to claim 1 or claim 2 wherein said second and third binding molecules brings about the precipitation of a precipitate on said electronic transducer by being each bound by a fourth binding molecule conjugated to an enzyme which catalyzes a reaction resulting in a precipitate on the electronic transducer.

8. A method according to claim 7 wherein said fourth binding molecule is an antibody.

9. A method according to claim 7 wherein said enzyme is selected from horseradish peroxidase, glucose oxidase or alkaline phosphatase.

10. A method according to claim 1 wherein said electronic transducer is selected from an electrode or a quartz crystal.

11. A method according to claim 10 wherein said precipitate is detected on said electronic transducer by a method selected from impedance spectroscopy or microgravimetric quartz crystal microbalance QCM analysis.

12. A method according to claim 1 wherein said chemical compound is a drug.

13. A method according to claim 1 wherein said compound reduces or inhibits the activity of the protease.

14. A method according to claim 1 wherein said compound increases the activity of the protease.

15. A method according to claim 1 wherein said protease is translated from an RNA sample.

16. A method according to claim 1 wherein said protease is a viral protease.

17. A method according to claim 16 wherein said viral protease is an HIV protease.

18. A method according to claim 17 wherein said compound is an anti-HIV protease inhibitor drug.

19. A method for detecting a drug-resistant virus, whose resistance is due to changes in a viral protease, comprising the method of claim 1 wherein the chemical compounds are anti-viral drugs, the protease is of viral origin, the substrate is a viral encoded polyprotein and the at least two segments of the substrate are individual viral proteins.

20. A method according to claim 19 wherein an extracted viral RNA sample is translated in the presence and in the absence of each of said anti-viral drugs.

21. A method for detecting a drug resistant protease comprising the method of claim 1 wherein the chemical compounds are drugs, the substrate is a polyprotein and the at least two segments of the substrate are individual proteins.

22. A method for following anti-viral drug resistance in a patient having a viral disease comprising:
(a) obtaining at a first time point a first virus-containing sample from said patient;
(b) analyzing the virus in the first sample for drug resistance according to the method of claim 19;
(c) obtaining at a second time point a second virus-containing sample from said patient;
(d) analyzing the virus in the second sample for drug resistance according to the method of claim 19; and
(e) comparing the drug resistance determined in steps (ii) and (iv).

23. A method for testing a chemical compound for its ability to inhibit the activity of a protease, said protease being capable of specifically cleaving a substrate into at least two segments, the method comprising:
(a) providing a specimen containing a protease, said specimen being divided into a sample A and a sample B.;
(b) providing an electronic transducer having bound thereto a first binding molecule capable of specifically binding a first segment of said substrate;
(c) providing a second binding molecule capable of specifically binding a second segment of said substrate, said second binding molecule being capable of bringing about the precipitation of a precipitate on said electronic transducer;
(d) providing a third binding molecule capable of specifically binding the first segment of said substrate at a site other than the site bound by the first binding molecule, said third binding molecule being capable of bringing about the precipitation of a precipitate on said electronic transducer,
(e) incubating said substrate with said sample A in the presence of said chemical compound and with said sample B in the absence of said chemical compound;
(f) dividing the mixture of the substrate with each of samples A and B from step (e) into two sub-samples, being sub-samples A(1) and A(2), and sub-samples B(1) and B(2);
(g) incubating said electronic transducer with each of the sub-samples of step (f);
(h) incubating the electronic transducer of step (g) which was incubated with each of sub-samples A(1) and B(1) in the presence of said third binding molecule;
(i) incubating the electronic transducer of step (g) which was incubated with each of sub-samples A(2) and B(2) in the presence of said second binding molecule;
(j) incubating the electronic transducer of steps (h) and (i) under conditions which bring about the precipitation of the precipitate on said electronic transducer; and
(k) measuring a signal from the electronic transducer indicating the presence or absence of the precipitate on said electronic transducer and calculating the ratio of signals obtained, a significant deviation from 1 in the ratio of A(2) to B(2), while the ratio of A(1) to B(1) is approximately 1, indicating that the activity of the protease has been inhibited by the chemical compound.

24. A method for identifying an anti-viral drug capable of reducing or inhibiting the activity of a viral protease, said protease being capable of specifically cleaving a viral polyprotein into at least two individual proteins, the method comprising:
(a) providing a speciman containing viral RNA, said specimen being divided into a sample A and a sample B;
(b) providing an electronic transducer having bound thereto a first binding molecule capable of specifically binding a first protein of said polyprotein;
(c)providing a second binding molecule capable of specifically binding a second protein of said polyprotein, said second binding molecule being capable of bringing about the precipitation of a precipitate on said electronic transducer;
(d) providing a third binding molecule capable of specifically binding the first protein of said polyprotein at a site other than the site bound by the first binding molecule, said third binding molecule being capable of bringing about the precipitation of a precipitate on said electronic transducer,
(e) subjecting said sample A to in vitro translation in the presence of said chemical compound and said sample B to in vitro translation in the absence of said chemical compound;
(f) dividing each of samples A and B from step (e) into two sub-samples, being sub-samples A(1) and A(2), and sub-samples B(1) and B(2), respectively;
(g) incubating said electronic transducer with each of the sub-samples of step (f);
(h) incubating the electronic transducer of step (g) which was incubated with each of sub-samples A(1) and B(1) in the presence of said third binding molecule;
(i) incubating the electronic transducer of step (g) which was incubated with each of sub-samples A(2) and B(2) in the presence of said second binding molecule;
(j) incubating the electronic transducer of steps (h) and (i) under conditions which bring about the precipitation of the precipitate on said electronic transducer; and
(k) measuring a signal from the electronic transducer indicating the presence or absence of the precipitate on said electronic transducer and calculating the ratio of signals obtained, the ratio of A(2) to B(2) being significantly greater than 1, while the ratio of A(1) to B(1) being approximately 1 indicating that the chemical compound may be effective as an anti-viral drug.

25. A kit for detecting chemical compounds having the ability to inhibit the activity of a protease, said protease being capable of specifically cleaving a substrate into at least two segments, the kit comprising: (1) an electronic transducer having bound thereto a first binding molecule capable of specifically binding a first segment of a protease substrate; and (2) a second binding molecule capable of specifically binding a second segment of said substrate, said second binding molecule being capable of bringing about the precipitation of a precipitate on said electronic transducer.
